(19) United States
(12) Patent Application Publication
(10) Pub. No.: US 2011/0269852 A1
(43) Pub. Date: Nov. 3, 2011

(54) METHODS AND MECHANISMS INVOLVING HYPERPIGMENTATION PARTICULARLY FOR AFRICAN AMERICAN SKIN

(75) Inventor: David H. McDaniel, Virginia Beach, VA (US)

(73) Assignee: Hampton University, Hampton, VA (US)

(21) Appl. No.: 13/099,290

(22) Filed: May 2, 2011

Related U.S. Application Data
(60) Provisional application No. 61/329,923, filed on Apr. 30, 2010.

(51) Int. Cl. C40B 30/04; A61P 17/00; A61K 45/00

(52) U.S. Cl. 514/789; 506/9

(57) ABSTRACT

Sets of genes are identified that show modulated activity in hyperpigmented sun-exposed (HE) and non-hyperpigmented sun-exposed (NHE) skin, when compared to non-hyperpigmented non-exposed (NHNE) skin. The modulated sets of genes reveal important information about the genetic changes that take place in skin as a result of environmental exposure and damage. The modulated sets of genes may be used to fabricate custom DNA microarrays for evaluating patients with skin diseases or disorders. The microarrays may also be used to screen new substances for treating skin diseases and disorders. The modulated gene sets, and substances that target them, may also be used to develop therapies for individuals who suffer from hypopigmentation, such as those with Fitzpatrick type I skin or vitiligo.

### CROSS-REFERENCE TO RELATED APPLICATIONS

This application claims priority under 35 U.S.C. §119(e) to provisional U.S. Patent Application No. 61/329,923, filed on Apr. 30, 2010, the disclosure of which is expressly incorporated by reference herein in its entirety

### BACKGROUND OF THE DISCLOSURE

1. Field of the Disclosure

The disclosure is directed to pigmentation disorders, particular hyperpigmentation disorders, and treatments thereof.

2. Related Art

Human skin tone can vary over a wide range of colors. It is believed that this variation evolved in response to competing demands on skin, which include protection from ultra-violet (UV) light and vitamin D production, that changed as early humans migrated from Africa to areas with different light intensities. Early attempts to classify skin tone were highly subjective and may have been influenced by racial considerations.

In contrast, modern classification schemes rely on objective measurements. For example, the Fitzpatrick scale divides skin color into six broad types based on UV response. Other researchers have measured the amount of light reflected from an area of skin that does receive much sun exposure, such as the underarm.

Skin color is produced by melanin in the skin. The basal layer of the epidermis contains melanocytes, which produce melanin from the amino acid tyrosine. This process is known as melanogenesis. Melanocytes store melanin in organelles known as melanosomes. Melanocytes form dendrites into the squamous layer, which is the layer of the epidermis above the basal layer, and use the dendrites to transfer melanosomes to keratinocytes in the squamous layer in response to UV damage to DNA. UV damage may also cause keratinocytes to signal melanocytes to proliferate, produce more melanin, or grow more dendrites to increase the transfer of melanin. Disruptions or misregulation of these signaling pathways can result in hyperpigmentation.

Hyperpigmentation is the darkening of an area of skin due to increased melanin. Solar lentigines, or liver spots, are one example of hyperpigmentation caused by abnormal signaling between keratinocytes and melanocytes. Hormonal changes can also result in hyperpigmentation. Melasma, or “mask of pregnancy,” is one example. Skin damage that produces inflammation may result in post-inflammatory hyperpigmentation (PIH). Damage can be from causes such as acne lesions, ingrown hairs, scratches, insect bites, wounding of the skin, and surfactant damage.

Current therapies for hyperpigmentation target melanin synthesis. Thus, these therapies affect all melanin production in a patient, which may result in increased UV sensitivity and other complications. By targeting the signaling pathways that produce hyperpigmentation, novel therapies can provide more specific treatments with fewer side effects. In addition, an understanding of the signaling pathways responsible for skin pigmentation can produce novel therapies, such as sunless tanning products that provide UV protection to people who sunburn easily and have difficulties tanning.

### SUMMARY OF THE DISCLOSURE

The disclosure meets the foregoing need and allows new therapies using signaling pathways, which results in a significant increase in treatment options and other advantages apparent from the discussion herein.

Accordingly, in one aspect of the disclosure a method for treating hyperpigmentation of the skin includes applying an effective amount of a substance to a hyperpigmented lesion. The substance may modulate signaling pathways in the cells in the hyperpigmented lesion. The method may be part of a method for preventing or treating one or more types of cancer of the skin when one or more of the signaling pathways is indicative of a cancerous or pre-cancerous state. The substance may include more than one substance, and the lesion may include more than one lesion. The substance may be applied topically in the form of a lotion, a shake lotion, a cream, an ointment, a gel, a transdermal patch, or a paste. The substance may be applied by intradermal injection or intracutaneous injection.

According to another aspect of the disclosure, a method for screening for at least one skin disease or condition includes providing a DNA microarray with a two or more fixed probes for a number of genes that are associated with one or more pathways known to be modulated in the skin disease or condition. The method also includes collecting a test sample and a control sample from a patient who is being screened, isolating RNA from both samples, performing microarray analysis with both samples, and identifying one or more pathways that are modulated in the test sample but not in the control sample. The identification of pathways produces a pattern of modulated pathways, which is compared to a pattern of modulated pathways associated with the skin disease or condition to produce a diagnosis.

In yet another aspect of the disclosure, a method for screening a substance for efficacy in treating a skin disease or condition includes identifying two or more sites on a patient with the skin disease or disorder. One or more sites acts as a test sites, and one or more sites acts as a control site. A first skin sample is collected from each site. The substance is applied one or more times to the test site but not to the control site. A second sample is collected from each site. Microarray analysis is used to identify gene sets that are modulated in the first test sample in comparison to the first control sample. Microarray analysis is further used to identify gene sets that are modulated in the second test sample in comparison to the second control sample. The first set of modulated genes is compared to the second set of modulated genes to determine the efficacy of the substance. The substance may be applied repeatedly to the test site over the course of two or more days.

Additional features, advantages, and embodiments of the disclosure may be set forth or apparent from consideration of the following detailed description, drawings, and claims. Moreover, it is to be understood that both the foregoing summary of the disclosure and the following detailed description are exemplary and intended to provide further explanation without limiting the scope of the disclosure as claimed.

### BRIEF DESCRIPTION OF THE DRAWINGS

The accompanying drawings, which are included to provide a further understanding of the disclosure, are incorporated in and constitute a part of this specification, illustrate embodiments of the disclosure and together with the detailed description serve to explain the principles of the disclosure. No attempt is made to shove structural details of the disclosure in more detail than may be necessary for a fundamental understanding of the disclosure and the various ways in which it may be practiced. In the drawings:

FIG. 1 shows the regulatory pathways that control melanogenesis.

### DETAILED DESCRIPTION OF THE DISCLOSURE

The embodiments of the disclosure and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure, which is defined solely by the appended claims and applicable law. Moreover, it is noted that like reference numerals represent similar parts throughout the several views of the drawings.

Identification of mechanisms underlying hyperpigmentation, particularly in African-American skin, begins by determining the difference between three classes of skin: hyperpigmented sun-exposed (HE) skin, non-hyperpigmented sun-exposed (NHE) skin, and non-hyperpigmented non-exposed (NHNE) skin from an area such as the upper underarm. Punch biopsies of 3 mm size were taken from each class of skin from each individual selected for the study. Subjects with post-inflammatory hyperpigmentation (PIH) were selected based on several criteria.

Subjects were limited to females between ages of 30 and 65 in good overall health with Fitzpatrick skin types IV, V, or VI and clinically-determined moderate to severe hyperpigmentation. Subjects were free of any systemic or dermatological disorder and have no known history of keloid formation, abnormal wound healing, or scarring. Subjects have not used a pigment lightening product in the past year. Subjects have no known hormonal disorders, are not currently using hormone therapy, and do not have any thyroid conditions or disorders. Subjects may be on birth control, but there must be no change in dosage for 12 months prior the study and no change in dosage during the study.

Potential subjects were excluded if they met at least one of the following conditions: (a) visible skin condition that could interfere with evaluations; (b) active atopic dermatitis, eczema, or psoriasis at test site; (c) excessive dryness or erythema at test site; (d) pregnant, planning pregnancy, or nursing during the study; (e) taking medications that could interfere with test results, including any regimen of steroidal/non-steroidal anti-inflammatory drugs, or antihistimines; (f) used topical retinoids, topical corticosteroids, topical antibiotics within the last 90 days; (g) used products containing salicylic acids, glycolics acids, alpha hydroxy acids, or high levels of vitamin C within the last 90 days; (h) used facial peels within the last 90 days; (i) used Accutane® of systemic steroids within the last 12 months; (j) received laser cosmetic treatments to the face within the last 12 months; (k) used a product designed to lighten or otherwise alter skin pigment, including hydroquinone and other bleaches, in the last 12 months; (l) currently taking a medication known to cause hyperpigmentation, such as minocycline; (m) currently enrolled in any other study or participated in a clinical study that involved the face, inner upper arm, or buttocks 28 days prior to start of current study; or (n) currently under treatment for asthma or type I diabetes.

The above criteria resulted in 3 female subjects with an average age of 39. Two subjects were of mixed African American and Native American ancestry, and the third had mixed African American and European ancestry. The average skin type of the subjects was Fitzpatrick V, which is described as sun-insensitive skin that rarely burns and tans well. Two subjects do not use sunscreen and one uses SPF 15. All subjects are non-smokers with no recorded medical allergies and no current medications. All subjects have never used pigment lightening products on the biopsy locations.

Each biopsy was split in half, with one part being used for histological analysis. RNA was extracted from the other half for microarray analysis. The histological analysis indicated that the average percentage of melanin in the samples is approximately equal across the three classes of skin, hyperpigmented sun-exposed (HE), non-hyperpigmented sun-exposed (NHE), and non-hyperpigmented non-exposed (NHNE). However, the melanocytes in the hyper-pigmented skin appear different than those in non-hyperpigmented skin. The hyperpigmented melanocytes appear darker, or more intensely stained, and more dendritic.

In the microarray analysis, three pair-wise comparisons were made for each subject: (1) a comparison of NHNE versus NHE, which demonstrates the changes in UV or environmentally exposed skin; (2) a comparison of NHE and HE, which demonstrates differences between adjacent hyperpigmented and non-hyperpigmented skin; and (3) NHNE and HE, which demonstrates the changes in hyperpigmented skin. Differentially expressed transcripts (DETs) were identified in each pair-wise comparison between skin classes. Identification criteria for DETs were an absolute fold-change of 1.8 or greater and a log ratio p-value of 0.001 or less. DETs from all three subjects were compiled, and Gene Ontology (GO) enrichment analysis was performed on the identified DETs to determine GO biological process categories over-represented by the set of DETs.

TABLE 1
Total number of DETs in each comparison

| Comparison | Skin Classes | Number of DETs |
|---|---|---|
| 1 | NHNE vs. NHE | 113 |
| 2 | NHE vs. HE | 279 |
| 3 | NHNE vs. HE | 1348 |

TABLE 2
Representative pathway analysis for comparison 2 (NHE vs. HE)

| | | Genes Differentially | Total Genes in | % of Pathway |
|---|---|---|---|---|
| GO Biological Process | P-Value | Expressed | Pathway | Affected |
| Immune response | 3.73E−05 | 13 | 653 | 2.0 |
| Cell motion | 4.91E−04 | 7 | 268 | 2.6 |
| Microspike assembly | 7.12E−04 | 2 | 8 | 25.0 |
| Negative regulation of gene | 7.12E−04 | 2 | 8 | 25.0 |
| expression, epigenetic | | | | |
| Regulation of transcription | 2.97E−03 | 2 | 16 | 12.5 |
| factor activity | | | | |
| Positive regulation of T cell | 3.35E−03 | 2 | 17 | 11.8 |
| proliferation | | | | |
| Skeletal system | 6.52E−03 | 5 | 228 | 2.2 |
| development | | | | |
| Regulation of T cell | 6.66E−03 | 8 | 536 | 1.5 |
| activation | | | | |
| Regulation of myeloid cell | 9.62E−03 | 2 | 29 | 6.9 |
| differentiation | | | | |
| Sensory organ development | 0.01018 | 1 | 2 | 50.0 |
| Inflammatory response | 0.01018 | 1 | 2 | 50.0 |
| Aromatic amino acid family | 0.01018 | 1 | 2 | 50.0 |
| catabolic process | | | | |
| Glutamate metabolic | 0.01523 | 1 | 3 | 33.3 |
| process | | | | |
| Response to antibiotic | 0.01523 | 1 | 3 | 33.3 |
| Negative regulation of cell | 0.01664 | 6 | 396 | 1.5 |
| proliferation | | | | |
| Blood circulation | 0.01946 | 3 | 111 | 2.7 |
| T cell activation | 0.01953 | 2 | 42 | 4.8 |

Comparison 2 (NHE vs. HE) was further examined to identify expression differences in genes known to play a role in the pigmentation or hyperpigmentation of skin regardless of mechanism. Genes involved in normal pigmentation generally showed relatively level expression. One exception is genes also known to be involved in hyperpigmentation, which were up-regulated in the hyperpigmented sample. A second exception is two genes, ET1/EDN1 and αMSH, which were both unexpectedly down-regulated in the hyperpigmented sample. ET1/EDN1 is a stimulator of melanocytes function, and αMSH is a keratinocytes-derived protein involved in regulating melanocytes proliferation or differentiation.

Similarly, genes involved in the tanning response showed level response except for those known to be involved in hyperpigmentation and one down-regulated gene, POMC, which activates the PKC signal cascade. PKC is up-regulated in this sample and known to be involved in hyperpigmentation, which may be related to the down-regulation of POMC. The pattern is also true for genes involved in photoaging, solar lentigines, hormonal hyperpigmentation, and post-inflammatory hyperpigmentation.

Certain genes not normally associated with hyperpigmentation were selected for further analysis. RAF1 is an oncogene that has been implicated in many cancers. RAF1 exhibits anti-apoptotic activity but is down-regulated in hyperpigmented skin, so it may promote cell death in this skin class. BCL2 is another anti-apoptotic oncogene that is down-regulated in hyperpigmented skin. BCL2 interacts directly with RAF1 as part of its anti-apoptotic function. KITLG, also known as SCF, encodes a ligand for the KIT tyrosine kinase receptor. The ligand is a cytokine involved in hematopoesis, spermatogenesis, and melanogenesis. SCF has been associated with pigment variations, including Familial Progressive Hyperpigmentation, hair color, and freckle density.

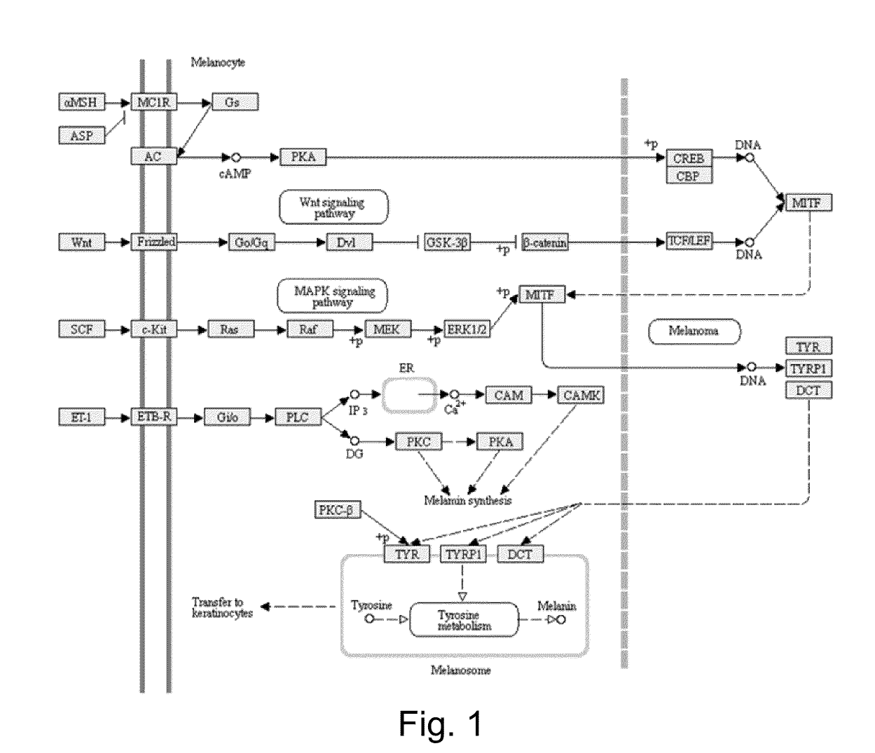

FIG. 1 shows the regulatory pathways that control melanogenesis. The expression analyses of the NHE vs. HE and NHNE vs. HE comparisons were pooled. The pooled data indicated that certain components of the melanogenesis signaling pathways are up- or down-regulated as would be expected in hyperpigmentation. In particular, CREB, SCF, TYRP1, PKC, and PKC-β change their expression levels as expected.

On the other hand, many components change expression levels in the opposite direction from what is expected. In other words, if a gene in this second group is expected to reduce its expression level, it instead increased its expression level. The pathway components in this group are c-Kit, RAF, ERK1/2, ET-1, ETB-K, and DCT.

The inhibition of RAF, which a major component in the MAPK signaling pathway, would be expected to lead to down-regulation of the downstream products, which include ERK1, ERK2, and TYRP1. ERK1 and ERK2 are indeed down-regulated, as expected, but TYRP1 is up-regulated.

This unexpected result suggests a feedback loop in the MAPK pathway. Similar feedback loops have recently been reported in the literature. In particular, RAF inhibitors have been found to hyper-activate RAF kinase activity, which suggests a feedback system driven by RAF itself. This feedback mechanism may be driven by alternate forms of RAF, known as ARAF, BRAF, and CRAF, RAF inhibitors may be more efficient at blocking one of these forms, which subsequently results in activation of an alternate form. Regardless of the exact feedback loop in hyperpigmentation, these results indicate that there may be a need for multiple inhibitors to effectively block the MAPK pathway and downstream signaling.

To gain additional insight into the mechanisms of hyperpigmentation, gene set analysis was performed on the pair-wise microarray results. The Gene Set Enrichment Analysis (GSEA) package was used to perform the analysis. GSEA is described in detail in Tamayo, at al. (2005, PNAS 102, 15545-15550) and Mootha, Lindgren, et al. (2003, Nat Genet 34, 267-273), both of which are expressly incorporated by reference herein in their entirety. Briefly, GSEA aims to identify gene sets with subtle but coordinated expression changes that cannot be detected by methods that only consider individual genes. Correlation-based expression changes emphasize the direction of expression change while not taking the magnitude of expression into account. This model more closely resembles the biological concept of co-expression.

GSEA determines whether members of a gene set are correlated with a sample type (e.g., hyperpigmented exposed (HE) skin). The gene set used in the present analysis is a set derived from the KEGG pathway database. KEGG is described in detail in Kanehisa at al. (2010, Nucleic Acids Res. 38, D355-D360), Kanehisa et al. (2006, Nucleic Acids Res. 34, D354-D357), and Kanehisa, et al. (2000, Nucleic Acids Res. 28, 27-30), all of which are expressly incorporated by reference herein in their entirety. For each gene set, GSEA outputs an enrichment score (ES), which indicates the correlation between the gene set and the sample; a normalized enrichment score (NES), which is the ES normalized across the analyzed gene sets; and a p-value, which is the statistical significance of the ES. GSEA does not differentiate between up-regulation and down-regulation; it can only assess differential expression of gene sets. Accordingly, the discussion below uses the term “modulation” rather than, e.g., suppression and activation.

TABLE 3
Modulated pathways in HE sample with p-value ≦ 0.05

Hsa04670: Leukocyte Transendothelial Migration
Hsa04060: Cytokine Cytokine Receptor Interaction
Hsa04514: Cell Adhesion Molecules
Hsa04650: Natural Killer Cell Mediated Cytotoxicity
Hsa04512: ECM Receptor Interaction
Hsa04612: Antigen Processing And Presentation
Hsa04940: Type I Diabetes Mellitus
Hsa04640: Hematopoietic Cell Lineage
Hsa04664: FcεRI Signaling Pathway
Hsa01032: Glycan Structures Degradation
Hsa00530: Aminosugars Metabolism
Hsa00511: N Glycan Degradation
Hsa00770: Pantothenate And CoA Biosynthesis
Hsa04660: T Cell Receptor Signaling Pathway
Hsa04620: Toll Like Receptor Signaling Pathway
Hsa05010: Alzheimer's Disease
Hsa01030: Glycan Structures Biosynthesis 1
Hsa04610: Complement And Coagulation Cascades
Hsa05120: Epithelial Cell Signaling In Helicobacter Pylori Infection
Hsa05110: Cholera Infection
Hsa05212: Pancreatic Cancer
Hsa00970: Aminoacyl tRNA Biosynthesis
Hsa04210: Apoptosis
Hsa00510: N Glycan Biosynthesis
Hsa03050: Proteasome
Hsa05030: Amyotrophic Lateral Sclerosis
Hsa04614: Renin Angiotensin System
Hsa04130: Snare Interactions In Vesicular Transport
Hsa04370: VEGF Signaling Pathway
Hsa04662: B Cell Receptor Signaling Pathway

TABLE 4
Modulated pathways in NHE sample with p-value ≦ 0.05

| | |
|---|---|
| Hsa01430: | Cell Communication |
| Hsa00350: | Tyrosine Metabolism |
| Hsa05217: | Basal Cell Carcinoma |
| Hsa00561: | Glycerolipid Metabolism |
| Hsa04530: | Tight Junction |
| Hsa04310: | Wnt Signaling Pathway |

TABLE 5
Modulated pathways in NHNE sample with p-value ≦ 0.05

| | |
|---|---|
| Hsa04080: | Neuroactive Ligand Receptor Interaction |
| Hsa00561: | Glycerolipid Metabolism |
| Hsa05217: | Basal Cell Carcinoma |

Thirty pathways in the exposed hyperpigmented sample (HE) show evidence of statistically significant, global modulation, i.e. broad-based, coordinated changes in gene expression, even if each particular gene isn't strongly or statistically significantly modulated. Six statistically modulated pathways were found in the NHE sample, while in the NHNE sample, only three pathways showed statistically significant modulation.

Melanin production does not appear to be among the most modulated pathways in any samples, including hyperpigmented skin. Despite this, tyrosine metabolism, which is required for melanin synthesis, is modulated in sun-exposed (NHE) skin, but not in armpit skin (NHNE).

The hyperpigmented skin (HE) itself exhibits statistically significant modulation of several pathways related to inflammation. These include leukocyte transendothelial migration, cytokine/receptor interactions, antigen processing and presentation, haematopoiesis, TCR signalling, TLR signalling, complement, and BCR signalling. These changes were not observed in either sun-exposed skin (NHE) or armpit skin (NHNE).

The basal cell carcinoma pathway did not show statistically significant modulation in the HE sample, but it was modulated in both the sun-exposed skin (NHE) and armpit skin (NHNE).

The hyperpigmented skin sample shows widespread modulation in a large number of cancer pathways, including colorectal cancer, pancreatic cancer, endometrial cancer, prostate cancer, bladder cancer, small cell lung cancer, non-small cell lung cancer, renal cell carcinoma, glioma, and melanoma, cell cycle regulation, p53 expression, and apoptosis regulation.

It is also immunogenic, showing modulation in a number of immune-related pathways. Modulated immune-related pathways include cytokine-cytokine receptor interaction, PI3K signaling, complement and coagulation cascades, antigen processing and presentation, TLR signaling, JAK-STAT signaling, NK cell mediated cytotoxicity, T cell receptor signaling, B cell receptor signaling, FcεRI signaling, leukocyte transendothelial migration, arachidonic acid metabolism, and PPAR signaling.

Cellular metabolism, including biosynthesis and degradation of cellular proteins, carbohydrates, lipids, and nucleic acids, in the hyperpigmented skin is highly abnormal and modulated. The HE samples show modulation in the metabolism of fructose, mannose, galactose, purines, pyrimidines, glutamate, cysteine, arginine, proline, tryptophan, aminophosphonate, selenoamino acid, glutathione, aminosugars, inositol phosphate, ether lipid, arachidonic acid, linoleic acid, sphingolipid and glycosphingolipid, butanoate, nicotinate and nicotinamide, porphyrin and chlorophyll, nitrogen, citrate, pentose phosphate, valine, leucine, isoleucine, folate, limonene, pinene, synthesis and degradation of both n- and o-glycans, pantothenate, coenzyme A, keratan, heparan, chondroitin, glycosylphosphatidylinositol, naphthalene, anthracene, benzoate, ethylbenzene, folate, carbon, aminoacyl tRNA, ribosomes, RNA polymerase, transcription factors, DNA polymerase, proteasomes, and PPAR signaling.

Cellular structure, as well as connection to and communication with nearby cells or extracellular matrix, also appears modulated. For example, the sample shows modulation of focal adhesion, extracellular matrix receptor interactions, cell adhesion molecules, and gap junctions. In addition, intracellular signal transduction is modulated. Affected signaling systems include ABC transporters, snare interactions in vesicular transport, PPAR signaling, MAPK signaling, ERBB signaling, MTOR signaling, VEGF signaling, p53 signaling, PI3K signaling, TLR signaling, JAK-STAT signaling, T cell receptor signaling, B cell receptor signaling, Fc epsilon RI signaling, insulin signaling, GNRH signaling, adipokine signaling, and epithelial signaling in H. pylori infection.

In addition, aspects of hormonal signaling are modulated, such as insulin, GNRH, and adipocytokine. Pathways typically associated with disease states or infections also exhibit modulation, including diabetes mellitus types I and II, Alzheimer's disease, Parkinson's disease, amyotrophic lateral sclerosis, Huntington's disease, cholera, Helicobacter pylori infection, and Escherichia coli infection.

Overall, this hyperpigmented skin (HE) sample is very different from the surrounding tissue (NHE) and from non-exposed tissue (NHNE). It exhibits modulation of several groups of pathways that are highly characteristic of cancer cells. These hallmarks are listed in Table 6, along with supporting pathway modulation observed in the HE sample.

TABLE 6
Common characteristics of cancer and observed modulation of pathways in HE samples

| Cancer | |
|---|---|
| Characteristic | Potential Pathway(s) |
| Self-sufficiency in | VEGF, GNRH |
| growth signals | |
| Insensitivity to anti- | Insulin signalling, GnRH signalling, Adipocytokine signalling, |
| growth signals | cytokine/receptor interactions, cell adhesion molecules |
| Tissue invasion and | Axon guidance, VEGF signalling, focal adhesion, ECM receptor |
| metastasis | interaction, cell , adhesion molecules, gap junctions, regulation of |
| | actin cytoskeleton |
| Limitless replicative | DNA polymerase, basal transcription factors, p53, protease, |
| potential | ribosomes, RNA polymerase, cell cycle |
| Sustained | VEGF, regulation of actin cytoskeleton |
| angiogenesis | |
| Evading apoptosis | Apoptosis, p53, cell cycle |
| Deregulated | Fructose, mannose, galactose, purines, pyrimidines, glutamate, |
| metabolism | cysteine, arginine, proline, tryptophan, aminophosphonate, |
| | selenoamino acid, glutathione, aminosugars, inositol phosphate, |
| | ether lipid, arachidonic acid, linoleic acid, sphingolipid and |
| | glycosphingolipid, butanoate, nicotinate and nicotinamide, porphyrin |
| | and chlorophyll, nitrogen, citrate, pentose phosphate, valine, leucine, |
| | isoleucine, glycan biosynthesis and degradation, folate, limonene, |
| | pinene, glycan synthesis and degradation (both n- and o- glycans), |
| | pantothenate, coenzyme A, keratan, heparan, chondroitin, |
| | glycosylphosphatidylinositol, naphthalene, anthracene, benzoate, |
| | ethylbenzene, folate, carbon, aminoacyl tRNA, ribosomes, RNA |
| | polymerase, transcription factors, DNA polymerase, proteasomes, |
| | PPAR signalling |
| Immuno-evasion | Cytokine-cytokine receptor interaction, PI3K signalling, complement |
| | and coagulation cascades, antigen processing and presentation, TLR |
| | signalling, JAK-STAT signalling, NK cell mediated cytotoxicity, T cell |
| | receptor signalling, B cell receptor signalling, Fc epsilon RI signalling, |
| | leukocyte transendothelial migration, arachidonic acid metabolism, |
| | PPAR signalling |
| Unstable DNA | DNA polymerase, basal transcription factors, p53 |
| Inflammation | Complement, TLR, JAK-STAT, NK cells, T cells, B cells, leukocyte |
| | migration, antigen processing; see also immuno-evasion |

Non-hyperpigmented sun-exposed skin (NHE) shows modulated metabolic pathways. The modulated pathways include glycolysis and gluconeogenesis, pentose and glucuronate, fatty acid, steroid biosynthesis, bile acid biosynthesis, androgen and estrogen biosynthesis, urea cycle and metabolism, alanine, aspartate, glycine, serine, threonine, methionine, histine, tyrosine, phenylalanine, gamma hexachlorocyclohexane, beta alanine, starch and sucrose, glycerolipid, alpha linolenic acid, pyruvate, 1- and 2-methylnapththalene, propanoate, 3-chloroacrylic acid, riboflavin, alkaloid, and xenobiotics.

Additional pathways were modulated in the NHE samples. Four pathways involved in organismal development displayed modulation: WNT signaling, dorso-ventral axis formation, notch signaling, and hedgehog signaling. In addition, pathways related to adherens junctions, tight junctions, and cell communications exhibited modulation. There was also modulation of olfactory and taste transduction pathways.

Genes involved in melanogenesis exhibited modulation, including the overall melanogenesis pathway as well as the tyrosine metabolic pathway. Only three cancer-related pathways were observed to be modulated in exposed skin: thyroid cancer, basal cell carcinoma, and acute myeloid leukemia.

Non-hyperpigmented non-exposed (NHNE) skin also showed evidence of modulated metabolic pathways. Modulation was evident in the pentose and glucuronate, androgen and estrogen, urea cycle and amino groups, analine, aspartate, arginine, proline, histidine, tyrosine, phenylalanine, glycerolipids, 1- and 2-methylnaphthalene, 3-chloroacrylic acid, limonene, pinene, alkaloid, and xenobiotic pathways. All of these pathways were also observed to be modulated in non-lesion, exposed skin (NLE) samples.

NHNE samples exhibited further similarities to the NHE samples. Three of the same pathways involved in organismal development were modulated in the non-exposed samples: WNT signaling, notch signaling, and hedgehog signaling. In addition, adherens junctions and tight junctions both showed modulation in NHNE and NHE samples. Olfactory, but not taste, signaling pathways were modulated in NHNE samples. Although the melanogenesis pathway was not modulated, the tyrosine metabolic pathway did display modulation.

There were no pathways that were uniquely modulated in NHNE skin (non-lesion, non-exposed); each of the 32 was also modulated in either NHE, skin or in HE skin. This is consistent with the role of NHNE as a control sample.

### Example 1

A DNA microarray is fabricated using genes encoding components of pathways associated with hyperpigmented or hypopigmented skin. Use of specific genes and pathways, as well as gene set analysis, allows the microarray to be used in the diagnosing of pigment disorders or predicting the response of such disorders to specific drugs or other therapies. The microarray may also be used to screen potential new drug and other therapies for the treatment of pigment disorders.

### Example 2

An effective amount of a substance is applied topically to the skin to prevent hyper-pigmentation. The substance may be applied prophylactically when hyperpigmentation may be expected to occur. For example, a person with a history of acne and subsequent hyper-pigmentation of healed pimples may apply the substance to pimples as they heal to deter hyper-pigmentation. A pregnant woman may apply the substance to her face to prevent or reduce hyperpigmentation as a result of hormonal changes. The substance may act, e.g., by blocking pathways associated with melanogenesis or dendrite formation.

### Example 3

An effective amount of a substance is applied topically to the skin to treat hyperpigmentation. The substance may be applied to lesions or areas of the skin that are hyperpigmented for any reason. The substance may contain a mixture of different active ingredients. The substance may activate signaling pathways in cells in the hyperpigmented region, thereby causing the cells to revert to an expression profile associated with non-hyperpigmented skin.

### Example 4

An effective amount of a substance is applied topically to the skin to treat or reverse damage to the skin from long-term exposure to ultraviolet light or other environmental factors. The substance may contain a mixture of different active ingredients. The substance may activate signaling pathways in cells in the damaged skin, thereby causing the cells to revert to an expression profile more closely resembling that of unexposed or undamaged skin.

### Example 5

An effective amount of a substance is applied topically to the skin to promote melanogenesis. The substance may be used to treat hypopigmentation resulting from, e.g., conditions such as vitiligo, scarring, or stretch marks. The substance may be applied to provide natural and long-lasting protection against ultraviolet light, including sunburns, to individuals with, e.g., Fitzpatrick type I, II, or III skin.

While the disclosure has been described in terms of exemplary embodiments, those skilled in the art will recognize that the disclosure can be practiced with modifications in the spirit and scope of the appended claims. These examples given above are merely illustrative and are not meant to be an exhaustive list of all possible designs, embodiments, applications or modifications of the disclosure.

What is claimed is:

1. A method for treating hyperpigmentation of the skin, the method comprising applying an effective amount of a substance to a hyperpigmented lesion.

2. The method of claim 1, wherein the substance modulates signaling pathways in cells in the hyperpigmented lesion.

3. The method of claim 1, wherein the substances comprises a plurality of substances.

4. The method of claim 1, wherein the hyperpigmented lesion comprises a plurality of hyperpigmented lesions.

5. The method of claim 1, wherein the substance is applied topically.

6. The method of claim 5, wherein the substance is in the form of a lotion, a shake lotion, a cream, an ointment, a gel, a transdermal patch, or a paste.

7. The method of claim 1, wherein the substance is applied by intradermal injection or by intracutaneous injection.

8. A method of preventing or treating at least one cancer of the skin, the method comprising the method of claim 2 wherein at least one of the modulated signaling pathways is indicative of a cancerous or pre-cancerous state.

9. A method of screening for at least one skin disease or condition, the method comprising:
providing a DNA microarray comprising a plurality of fixed probes for a plurality of genes, the plurality of genes associated with at least one pathway known to be modulated in the at least one skin disease or condition;
collecting a test sample and a control sample from a patient to be screened for the at least one skin disease or condition;
isolating RNA from the test sample and from the control sample;
performing a DNA microarray analysis using the isolated RNA;
identifying at least one pathway that is modulated in the test sample but not in the control sample, the identification resulting in a pattern of modulated pathways; and
comparing the pattern of modulated pathways to a pattern of modulated pathways in the at least one skin disease or condition, the comparison resulting in a diagnosis.

10. A method of screening a substance for efficacy in treating a skin disease or disorder, the method comprising:
identifying at least two sites on a patient with the skin disease or disorder, the at least two sites comprising a test site and a control site;
collecting a first skin sample from each of the at least two sites on the patient with the skin disease or disorder, the samples comprising a first test sample and a first control sample;
applying the substance at least once to the test site but not to the control site;
collecting a second skin sample from each of the at least two sites on the patient with the skin disease or disorder, the samples comprising a second test sample and a second control sample;
using microarray analysis to identify modulated gene sets in the first test sample as compared to the first control sample, resulting in first modulated gene sets;
using microarray analysis to identify modulated gene sets in the second test sample as compared to the second control sample, resulting in second modulated gene sets;
comparing the first modulated gene sets with the second modulated gene sets to determine an efficacy of the substance.

11. The method of claim 10, further comprising applying the substance to the test area repeatedly over a plurality of days.
